(19) United States
(12) Patent Application Publication
(10) Pub. No.: US 2013/0018425 A1
(43) Pub. Date: Jan. 17, 2013

(54) SYSTEM AND METHODS FOR IN VIVO ADJUSTABLE BONE PLATE

(71) Applicants: Edward Seldin, Cambridge, MA (US); Alexander H. Slocum, Cambridge, MA (US)

(72) Inventors: Edward Seldin, Cambridge, MA (US); Alexander H. Slocum, Cambridge, MA (US)

(73) Assignee: The General Hospital Corporation, Boston, MA (US)

(21) Appl. No.: 13/635,707

(22) PCT Filed: Mar. 24, 2011
(86) PCT No.: PCT/US11/29770

§ 371 Date: Sep. 18, 2012

Related U.S. Application Data
(60) Provisional application No. 61/317,884, filed on Mar. 26, 2010.

(51) Int. Cl. A61B 17/80

(52) U.S. Cl. 606/286

(57) ABSTRACT

A bone plate and method in vivo deployment is designed to secure proximate portions of a bone together. The bone plate includes first and second anchoring regions extending in at least two direction to form a surface configured to extend coaxially with a bone and having formed therein a plurality of passages configured to receive fasteners to secure the first and second anchoring region to respective first and second portions of the bone. A deformation region extends to join the first and second anchoring regions along at least one plane extending substantially co-planar with the surface of the first anchoring region and the surface of the second anchoring region and having reduced structural integrity with respect to the first and second anchoring region to deform the deformation region and, thereby, adjust a relative orientation between the first and second anchoring regions in response to forces applied to the deformation region.

### CROSS-REFERENCE TO RELATED APPLICATIONS

This application is based on, claims the benefit of, and incorporates herein by reference, U.S. Provisional Patent Application Ser. No. 61/317,884, filed on Mar. 26, 2010, and entitled “Bone Plate.”

### STATEMENT REGARDING FEDERALLY SPONSORED RESEARCH

N/A.

### BACKGROUND OF THE INVENTION

The present application is directed to bone plate systems and methods of clinical use and, more particularly, a system and method for using a bone plate configured to facilitate improved alignment and contact between adjacent portions of bone before and, in particular, after the bone plate is affixed to the adjacent portions of bone.

Bones provide many mechanical and biological functions, including support of organs and muscles for movement and support, protection of organs, and generation of blood cells. While generally structurally sound, damaged bones, particularly those with substantial fractures or brakes may require structural reinforcement to facilitate repair. Though these structural reinforcement systems have various characteristics and clinical indications of use, common reinforcement systems are generally designed to maintain the position of the bone and alignment of portions of the bone over an extended duration to facilitate healing in a desired manner. Common bone reinforcement systems include systems located externally to the body, such as casts, splints, and the like, and systems that are designed to be arranged internally to the body, such as screws, pins, and bone plates.

Externally-located bone reinforcement systems and internally-located bone reinforcement systems have clear physical differences and, likewise, have different clinical indications for use. For example, due to the non-interventional nature of externally-located bone reinforcement systems, externally-located bone reinforcement systems are preferable in a wide range of clinical situations. Accordingly, internally-located bone reinforcement systems are often reserved for use in treating severe traumas or when subtle or exacting control of the bone is required to facilitate proper healing.

Bone plates are one common resource in forming internally-located bone reinforcement systems. Bone plates, and associated devices, such as screws and pins, are devices that are specifically designed to be use during a surgical procedure to be attached directly to bone. In one common use, bone plates are used to position adjacent portions of bone proximate to a brake or fracture to secure the bone in desired position through the duration of the healing process. For example, a bone plate can be used to hold two separate bone fragments together while they heal and grow back together.

Referring now to FIGS. 1A and 1B, a traditional bone plate 10 system includes a contiguously-rigid, substantially-planar plate 12, which is commonly made from titanium, stainless steel, or another rigid material that is suitable for implantation within a patient. Arranged along a planar surface 14 of the plate 12 is a series of holes 16.

In clinical use, the plate 12 is designed to be affixed to a bone 18 through pins or screws 20 that extend through the holes 16 and secure the plate 12 to the bone 18. However, when solid objects with complex surface features, for example, including convexities and concavities, are brought into juxtaposition, three nonlinear points of contact 22, 24, 26 between the bone 18 and the bone plate 12 define a plane of contact 28 that confers a measure of mechanical stability between the bone plate 12 and the bone 18.

In many clinical settings, it is unusual for such objects to have more than three initial points of contact 22, 24, 26. This geometrical truism accounts for the fact that in all but the most unusual of circumstances, fitting a bone plate 12 to the surface of a bone 18 creates voids 30 between the plate 12 and the underlying bone 18 that are formed between the points of contact 22, 24, 26. As illustrated, the bone plate 12 may readily traverse such voids 30 when affixed to the bone 18 through the screws 20.

While these voids 30 may be of reduced consequence is many clinical settings, such as bulk orthopedic repairs of large bones having suffered substantial trauma, in a variety of clinical settings it may be advantageous to control the number and size of the voids 30. Accordingly, many clinicians will look to modify the bone plate 12 prior to positioning of the bone plate 12 for fixation to the bone 18. Though traditional bone plates 12 are highly rigid and designed to be very structurally sound, clinical resources may be used to bend or otherwise modify the bone plate 12 prior to positioning of the bone plate 12 for fixation to the bone 18. However, since such modifications are performed prior to positioning of the bone plate 12 for fixation to the bone 18 and with the bone plate 12 located remotely from the bone 12, such as secured in a vice or other tool to facilitate the desired modification, the extent of the modifications are ultimately little more than educated guess work and limited to substantial or bulk modifications of the overall geometry of the bone plate 12. To further complicate the variables managed by the clinician, when finally securing the bone plate 12 using the screws 20, tight application of the screws 20 can lead to deformation of the bone plate 12 and deflection of the segments of the bone 18 across the line of a fracture. The size accordingly, despite efforts to minimize the size and extent of voids 30 formed when affixing the bone plate 12 to the bone 18 by modifying the bone plate 12 prior to positioning of the bone plate 12 for fixation to the bone 18, the size of the resulting voids 30 and the potential for deflection of fracture segments in application of screws is a readily correlated to the mechanical skill, prior experience, and guess-work of the clinician.

Relying on the mechanical skill, prior experience, and guess-work of the clinician may be suitable in a variety of situations; however, when subtle control, rather than bulk alignments, is paramount, such as, for example, in craniomaxillofacial surgery, voids and deflection of fracture segments that would otherwise be tolerable in bulk orthopedic repair, can have highly-undesirable consequences. In some circumstances, it may be necessary to remove and re-attach a bone plate having even subtle undesirable fitting to avoid substantial structural consequences. For example, when repairing a patient's jaw, subtle miss-fittings of a bone plate can result in miss-alignments of the patient's teeth that cause substantial discomfort and the need for orthodontic intervention. However, removal of a misaligned or undesirably aligned bone plate for subsequent re-attachment results in substantial physical trauma and even structural weakening of the bone, which is directly contrary to the clinical purpose of the interventional procedure, as holes are reformed in the bone and the bone plate is re-affixed thereto. Responsive thereto, some bone plate systems have been developed with holes having chamfered or angled edges, such that loosening and/or tightening of fasteners in the holes can provide a small amount of positional adjustment between the adjacent bone portions. However, these bone plate systems still require substantial guess work by the clinician prior to positioning and affixing the bone plate to the bone.

In an effort to reduce the general guess work associated with performing modifications to the bone plate prior to positioning and affixing the bone plate, referring to FIG. 2, some bone plate systems 40 have been designed to include a rigid hinge 42 designed to divide the bone plate system 40 into two separate, rigid planes 44, 46 secured by the rigid hinge 42. While such bone plate systems 40 provide a mechanism that facilitates adjustments to the overall bone plate system 40 by allowing the clinician to more readily alter the overall structural configuration of the bone plate system 40 by bending the rigid hinge 42, this adjustability comes with reduced structural integrity of the overall bone plate system 40 and encourages a substantially limited range of adjustability that further exacerbates the reduced structural integrity of the overall bone plate system 40. That is, the rigid hinge 42 divides the bone plate system 40 into the aforementioned rigid planes 44, 46 that can be moved along a rotational arc 48 formed perpendicular to (in plane) and transverse to (out of plane) with an axis of rotation 50 formed by the rigid hinge 42. Unfortunately, as illustrated, since this axis of rotation 50 is generally aligned with the very fracture or other trauma to the bone that is to be secured by the bone plate system 40, the reduced structural integrity formed by the rigid hinge 42 can undermine the clinical purpose of bone plate system 40. Further still, the rigid hinge 42 substantially changes the profile of the bone plate system 40 by forming a raised portion that may be undesirable or unsuitable for some applications and, particularly, many applications that call for increased flexibility and subtlety in the instillation, such as when repairing the jaw.

Therefore, it would be desirable to provide a bone plate that facilitates a clinical adjustments and, particularly, clinical adjustments desirable in clinical applications dictating subtle adjustments, without sacrificing structural integrity ore creating undesirable plate profiles.

### SUMMARY OF THE INVENTION

The present invention overcomes the aforementioned drawbacks by providing an adjustable bone plate and associated methods that is capable of supporting or reinforcing a damaged area of bone, and facilitates optional adjustment of positions and/or orientations of adjacent bone portions in a plurality of modes and/or directions after the bone plate is affixed to the target bone thereby substantially reducing the need for trial-and-error methods or other guesswork in properly deploying the bone plate.

In accordance with one aspect of the invention, a method of setting a fracture in a bone is disclosed that includes securing a bone plate to portions of a bone on opposing sides of the fracture in the bone by affixing a plurality of fasteners through respective passages in the bone plate and into bone on the opposing sides of the fracture. The method also includes, with the bone plate secured to the bone, deforming at least one of a pair of bridge arms forming a central portion of the bone plate extending between portions, of the bone plate affixed by the plurality of fasters on the opposing sides of the fracture to position the opposing sides of the fracture proximate one another to extend coaxially across the fracture.

In accordance with another aspect of the invention, a bone plate system is disclosed that is configured to be adjusted in vivo to secure proximate portions of a bone together. The bone plate system includes a first anchoring region extending from a first end to a second end to form a substantially planar surface configured to extend coaxially with a first surface of a bone and having formed therein a plurality of passages. A first plurality of fasteners is configured to extend through the plurality of passages formed in the first anchoring region to secure the first anchoring region to the first surface of the bone. The bone plate system also includes a second anchoring region extending from a first end to a second end to form a substantially planar surface configured to extend coaxially with a second surface of the bone and having formed therein a plurality of passages. A second plurality of fasteners is configured to extend through the plurality of passages formed in the second anchoring region to secure the second anchoring region to the second surface of the bone. The bone plate system further includes a deformation region including a first bridging arm and a second bridging arm extending in a parallel, spaced relation from the second end of the first anchoring region to the second end of the second anchoring region to arrange the first anchoring region and the second anchoring region in a spaced relation and define a plane that is substantially co-planar with the first surface and the second surface. The first bridging arm and the second bridging arm are configured to deform from the parallel, spaced relation forming the plane that is substantially co-planar with the first surface and the second surface after the first anchoring region is secured to the first surface of the bone by the first plurality of fasteners and the second anchoring surface is secured to the second surface of the bone by the second plurality of fasteners to perform simultaneous adjustments of the bone plate system and relative positions of the first surface of the bone and the second surface of the bone with respect to a fracture formed in the bone between the first surface of the bone and the second surface of the bone.

In accordance with yet another aspect of the invention, a bone plate is disclosed that is configured to be deployed in vivo to secure proximate portions of a bone together. The bone plate includes a first anchoring region extending in at least two direction to form a surface configured to extend coaxially with a bone and having formed therein a plurality of passages configured to receive fasteners to secure the first anchoring region to a first portion of the bone. The bone plate also includes a second anchoring region extending in at least two direction to form a surface configured to extend coaxially with the bone and having formed therein a plurality of passages configured to receive fasteners to secure the second anchoring region to a second portion of the bone. The bone plate further includes a deformation region extending to join the first anchoring region and the second anchoring region along at least one plane extending substantially co-planar with the surface of the first anchoring region and the surface of the second anchoring region and having reduced structural integrity with respect to the first anchoring region and the second anchoring region to deform the deformation region and, thereby, adjust a relative orientation between the first anchoring region and the second region in response to forces applied to the deformation region.

These and other features and advantages of the present invention will become apparent upon reading the following detailed description when taken in conjunction with the drawings.

### BRIEF DESCRIPTION OF THE DRAWINGS

FIGS. 1A and 1B are a perspective and side elevational view of a prior-art bone plate and exemplary clinical setting;

FIG. 2 is an illustration of another prior-art bone plate including a rigid hinge and exemplary clinical setting;

FIG. 3 is a perspective view of a bone plate in accordance with the present invention;

FIGS. 4A and 4B are plan views of a bone plate in accordance with the present invention illustrated in an initial configuration and in an exemplary deformed configuration;

FIGS. 5A and 5B are plan views of a bone plate in accordance with the present invention illustrating in an initial configuration and in another exemplary deformed configuration;

FIGS. 6A and 6B are perspective views of a bone plate in accordance with the present invention illustrated in an initial configuration and in yet another exemplary deformed configuration;

FIG. 7 is a plan view of a series of deformation regions in accordance with the present invention;

FIG. 8 is plan views of a bone plate in accordance with the present invention; and

FIG. 9 is an illustration of an exemplary tool for use to controllably deform a bone plate in accordance with configurations of the present invention.

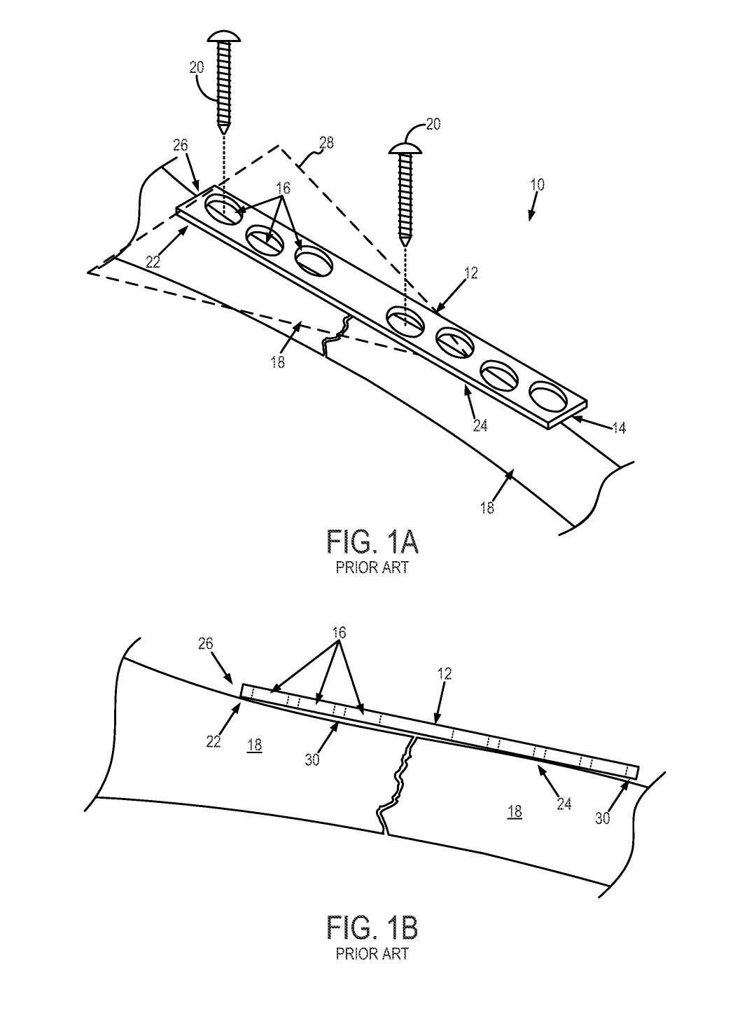

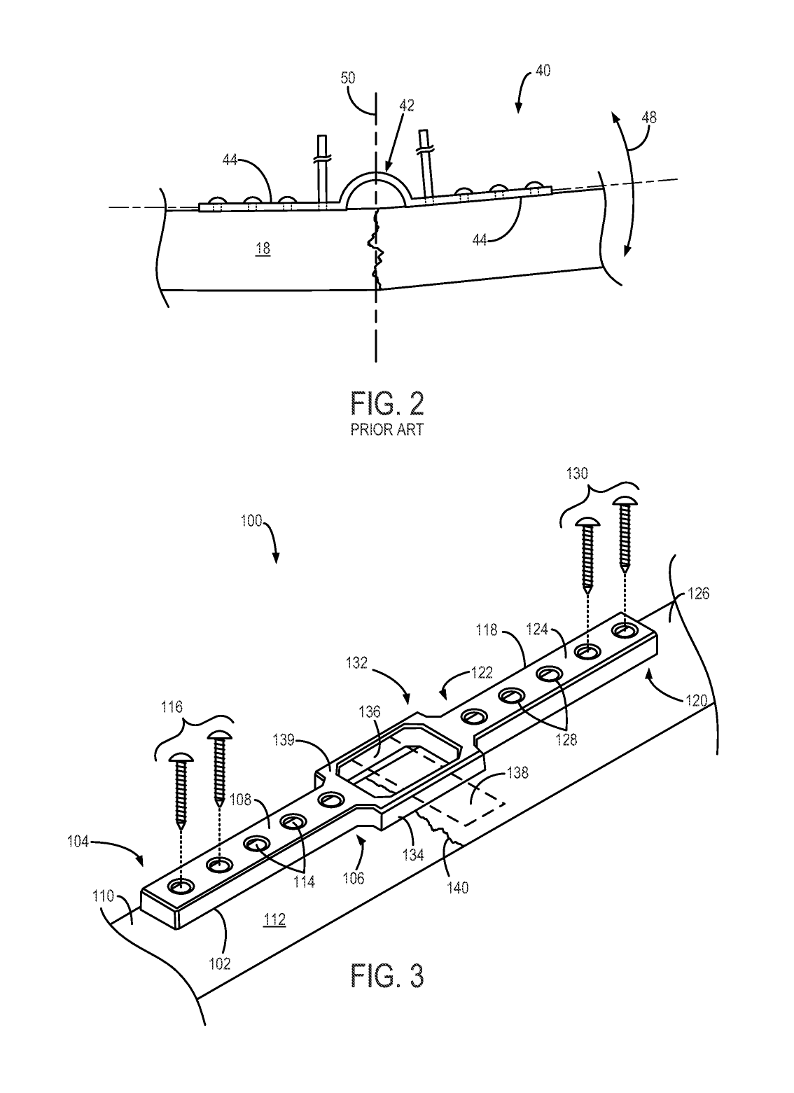

### DETAILED DESCRIPTION OF THE INVENTION

Referring now to FIG. 3, a perspective view of a bone plate system 100 in accordance with the present invention is shown. The bone plate system 100 includes a first anchoring region 102 extending from a first end 104 to a second end 106 to form a substantially planar surface 108 configured to extend coaxially with a 110 surface of a bone 112. The first anchoring region 102 has formed therein a plurality of passages 114 configured to receive a first plurality of fasteners 116 configured to extend through the plurality of passages 114 to secure the first anchoring region 102 to the surface 110 of the bone 112. The plurality of passages 114 may or may not be threaded. Also, the plurality of fasteners 116 may include screws, pins, wires, and the like.

The bone plate system 110 also includes a second anchoring region 118 extending from a first end 120 to a second end 122 to form a substantially planar surface 124 configured to extend coaxially with a second surface 126 of the bone 112. The second anchoring region 118 includes a plurality of passages 128 configured to receive a second plurality of fasteners 130 configured to extend through the plurality of passages 128 formed in the second anchoring region 118 to secure the second anchoring region 118 to the second surface 126 of the bone 112.

The plurality of passages 128 may or may not be threaded. The size and distribution of the passages 128 in can be selected and configured based on the bone 112 it is to be affixed to, the type of fastener used, and the like. In general, each anchoring region includes at least two holes to facilitate attachment to the underlying portion of bone in a fixed orientation. Other attachment provisions may be provided on the anchoring regions instead of or in addition to holes. For example, notches or grooves may be provided in the anchoring region if wires are used to affix the bone plate 100 to a bone 112.

Furthermore, the plurality of fasteners 130 may include screws, pins, wires, and the like. Bone screws may include unicortical screws that penetrate the bone cortex once, adjacent to the bone plate 100, and/or bicortical bone screws that pass through the entire bone 112, including the cortex located on the side of the bone opposite the bone plate 100. Such cortical screws can be used for affixing the bone plate 100 to a harder region of bone, such as to a shaft portion of a bone. Cancellous bone screws, which have larger threads than cortical screws, can also be used to affix the bone plate to softer portions of bone, such as near the ends of a long bone.

The bone plate system 110 also includes a deformation region 132 including a first bridging arm 134 and a second bridging arm 136 extending in a parallel, spaced relation from the second end 106 of the first anchoring region 102 to the second end 122 of the second anchoring region 118 to arrange the first anchoring region 102 and the second anchoring region 118 in a spaced relation and define a plane 138 that is substantially co-planar with the first surface 108 and the second surface 124. As will be described in detail, the deformation region 132 has reduced structural integrity with respect to the first anchoring region 102 and the second anchoring region 118, if only due to the reduced surface area of the deformation region 132 when compared to the anchoring regions 108, 118, to thereby be advantageously deformed even after the bone plate system 100 has been affixed the bone 112.

As will be described in further detail, the first bridging arm 136 and the second bridging arm 138 are configured to deform from the parallel, spaced relation forming the plane 138 that is substantially co-planar with the first surface 108 and the second surface 124 illustrated in FIG. 3. More particularly, the first bridging arm 136 and the second bridging arm 138 are configured to be deformed the after the first anchoring region 102 is secured to the first surface 110 of the bone 112 by the first plurality of fasteners 116 and the second anchoring surface 118 is secured to the second surface 126 of the bone 112 by the second plurality of fasteners 130 to perform simultaneous adjustments of the bone plate system 100 and relative positions of the first surface 110 of the bone 112 and the second surface 126 of the bone 110 with respect to a fracture 140 formed in the bone 112 between the first surface 110 of the bone 112 and the second surface 126 of the bone 112. It is noted that the bone plate system and, specifically, the deformation region 132 may be designed, such that the aforementioned deformational forces may be counteracted (balanced or neutralized) within the deformation region 132, with no force of significance being transmitted to the attached bone in the process.

The bone plate system 100 can be shaped to avoid or reduce irritation to the bone and surrounding soft tissue. For example, the bone plate system 100 may have a low profile to reduce protrusion into adjacent tissue. It can also be provided with smooth, rounded, chamfered, and/or burr-free surfaces and edges. Other materials that may be used to form conventional bone plates can also be used in configurations of the present invention.

A method for using the above-described bone plate system 100 includes exposing the fractured or damaged area 140 of bone 112 using surgical techniques to displace overlying skin, muscle, and other tissue. The anchoring regions 102, 118, as described, are designed to be affixed to the bone 112 about the discontinuity or damaged area 140 using the fasteners 116, 130.

Unlike traditional bone plates, after the anchoring regions 102, 118 are attached to the bone 112, the bone plate system 110 may be advantageously adjusted along with the relative positions of the bone portions on opposing sides of the fracture 140. Some adjustments may include alterations of the angular orientation between the bone portions, and/or provide, increased, or reduced compressive stress to the fracture 140. As will be described, a wide variety of adjustments can be made by manipulating or deforming the deformation region 132 of the bone plate system 100.

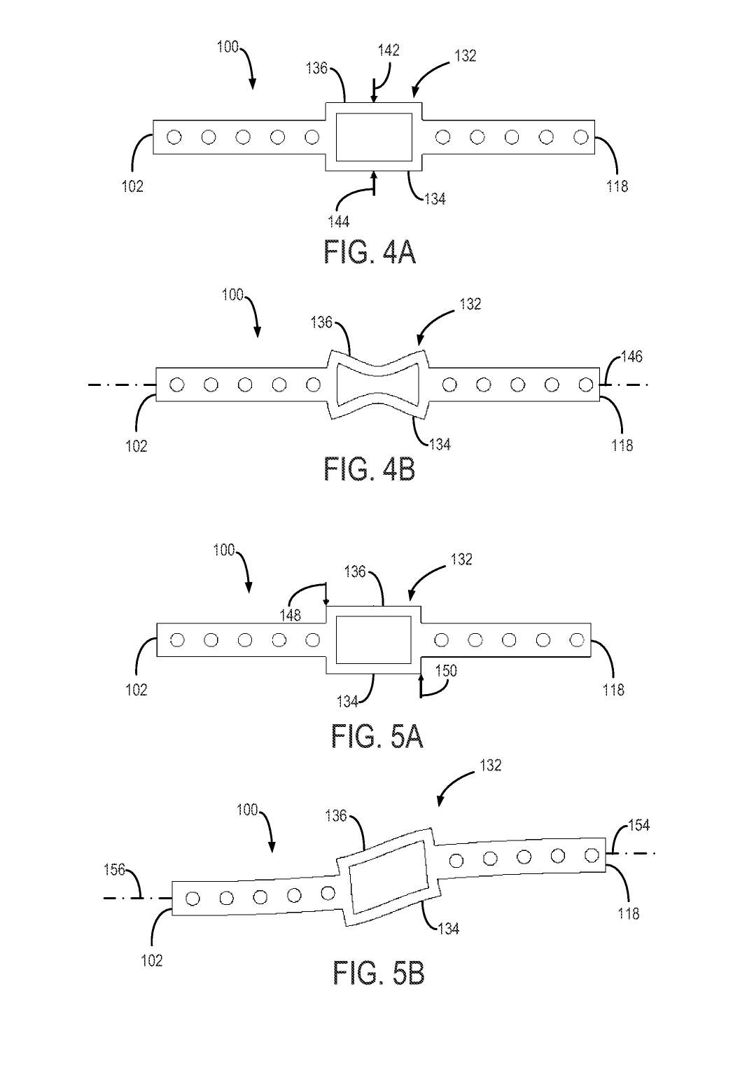

For example, referring to FIGS. 4A and 4B, a compressive force, such as indicated by arrows 142, 144 in FIG. 4A, can be applied to opposite sides of the deformation region 132. The force is preferably sufficient to provide a plastic deformation in the deformation region 132, as shown in FIG. 4B. This deformation tends to pull the anchoring regions 102, 118 towards each other. When the anchoring regions 102, 118 of the bone plate system 100 are each affixed to a bone, such deformation can pull the bone portions towards each other and/or create or increase a compressive stress where the bone portions meet, such as at a fracture or break in the bone. The direction of movement or compressive stress force resulting from this deformation can be along a longitudinal axis 146 of the bone plate system 100 if the deformation is performed symmetrically. The amount of plastic deformation of the deformation region 132 may be greater or less than that shown in FIG. 4B, and can be controllably generated to achieve a particular amount of motion and/or compressive stress between the bone portions to which the bone plate system 100 is affixed.

It is contemplated that the present invention can provide a bone plate system that exhibits, among other modes, the deformation behavior shown in FIG. 4B. This behavior results from the geometric characteristics of the deformation region 132 and the forces applied and tools used to apply to such forces to the bone plate system. By applying a compressive force to a central portion of the deformation region 132, ends of the bone plate can be brought closer together. Thus, the present invention can provide a bone plate that facilitates generation of a compressive force between adjacent portions of bone, which can assist in the healing process, through application of a compressive force to the deformation region 132, such as illustrated in FIGS. 4A and 4B.

A wide variety of further exemplary adjustment can be provided using the present invention. For example, referring to FIGS. 5A and 5B, a force may be applied to the deformation region 130 corresponding to arrows 148, 150. This force 148, 150 can be sufficient to generate a plastic deformation of the deformation region 132 to create the configuration illustrated in FIG. 5B. This deformation tends to shift the alignment of the anchoring regions 102, 118 as illustrated in FIG. 5B. For example, a longitudinal axis 152, 154 of each anchoring region 102, 118, respectively, is shifted laterally, to produce an offset in the axes 152, 154. A corresponding lateral shift can thus be produced in the bone portions to the anchoring regions 102, 118 are affixed. The amount of such shift can be controlled or determined based on the amount of deformation achieved by the deformation region 132.

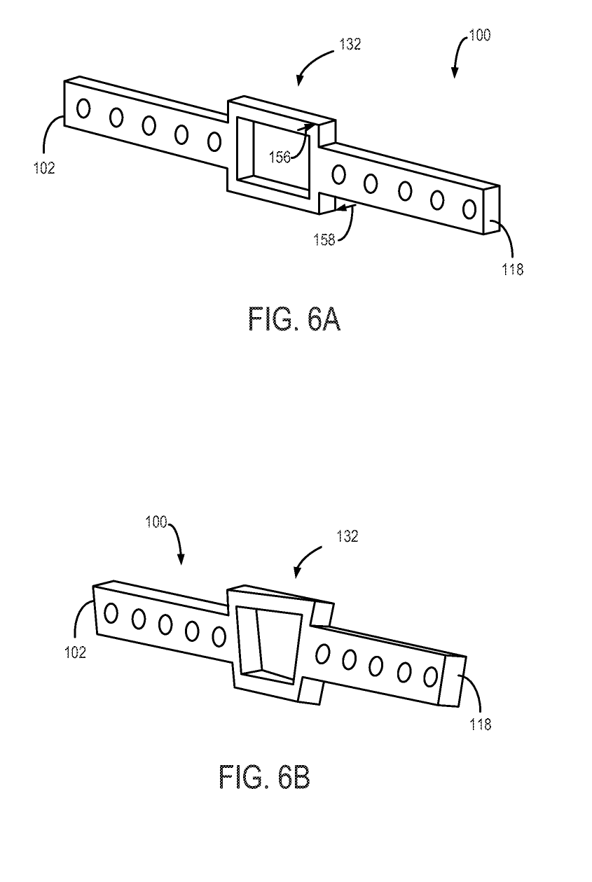

Referring now to FIGS. 6A and 6B, a force, as indicated by arrows 156, 158 can be applied to portions of the deformation region 132 proximal to a particular anchoring region, in the illustration, the second anchoring region 118. This force represented by arrows 156, 158 can be sufficient to generate a plastic deformation of the deformation region 132, such as illustrated in FIG. 6B. This deformation tends to provide a rotational shift, such as a twisting displacement, between the anchoring regions 102, 118, and thus between bone portions to which the anchoring regions 102, 118 are affixed.

Many similar and different deformations are contemplated. For example, a deformation may be created to from a “collapse” of one side of the deformation region 132 and can provide an angular shift between the longitudinal axes of the anchoring regions 102, 118 that lies substantially in the plane of the bone plate. Also, more complex types of adjustment can be provided by applying forces to particular locations of the deformation region 132 to generate a plastic deformation in the deformation region 132, such as an offset, a rotation, and multi-dimensional combinations of deformations between the anchoring regions 102, 118

Each anchoring region 102, 118 may have an elongated rectangular shape or other shapes may be provided based on the type of bone to which and clinical application in which the bone plate system 10 is to be attached. For example, an anchoring region may be provided with a non-uniform width, an angled or otherwise non-linear shape as viewed from above, and the like. The bottom surface of an anchoring region may be substantially flat, or it may optionally be contoured, for example, curved, to better conform to the surface shape of a region of bone to which it will be affixed.

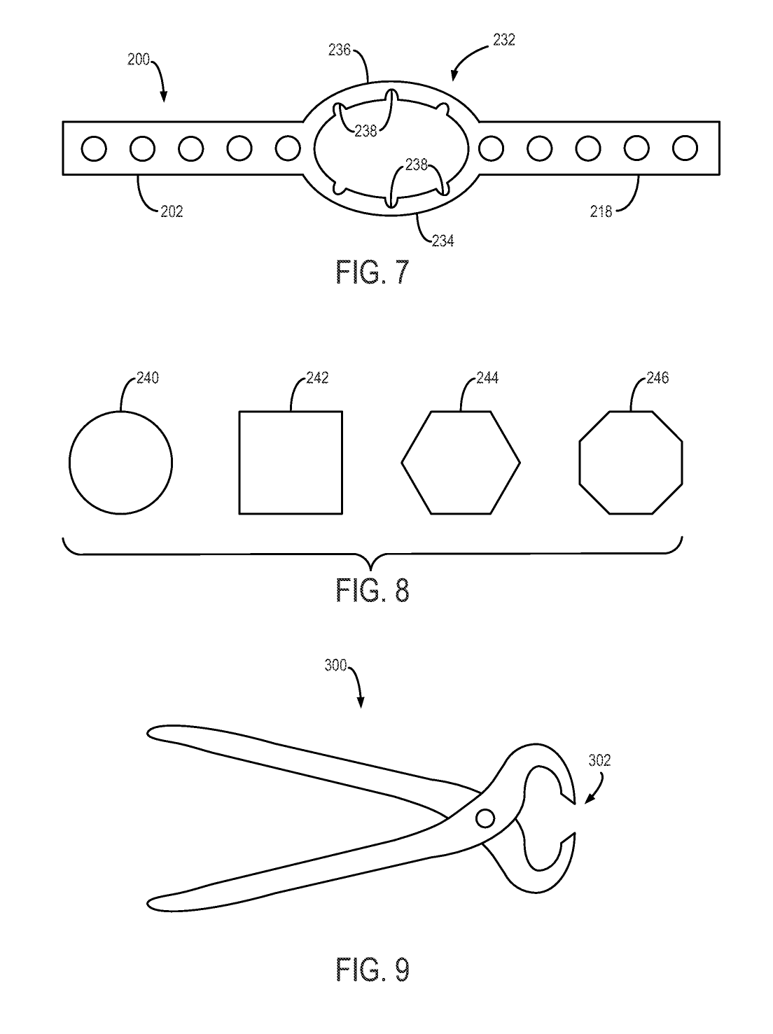

Furthermore, it is contemplated that the deformation region 132 of the bone plate system 100 may take a variety of forms and, more particularly, that the bridge arms 134, 136 may be configured to form a variety of geometric shapes through the above-described, spaced relation in combination with the anchoring regions 102, 118. For example, referring to FIG. 7, a bone plate 200 can be provided as described herein, where the deformation region 232 extends between anchoring regions 202, 218 in a shape that is substantially oval or elliptical. In this case, bridge arms 234, 236 are formed in an actuate path. A wide variety of other configurations are contemplated. For example, referring to FIG. 8, a deformation region may be formed as a circle 240, square 242, octagon 244, hexagon 246, other trapezoid, or a wide variety of other shapes.

In a further configurations, portions of the deformation region proximal to the anchoring regions can be reinforced, thickened, stiffened, or the like. This reinforcement can reduce the amount of deformation that may occur in these portions of the deformation region relative to that produced in the central portion of the deformation region when a force is applied thereto. For example, referring to FIG. 3, reinforcements 141 can be provided to achieve a more localized and/or controllable deformation of the deformation region when a force is applied thereto. Portions of the deformation region can also be reinforced in other ways, for example, by forming the reinforced portions from a stronger or stiffer material, by providing different heat treatments or other processing steps to different portions of the deformation region, or the like.

Similarly to reinforcements, additional structural weaknesses may be formed about the deformation region. For example, referring again to FIG. 7, a plurality of indentations 238 may be formed, for example, on the interior of the deformation region 232. Of course, exteriorly located and other locations of indentations, or protrusions, are contemplated. However, as illustrated in FIG. 7, the indentations 238 may be designed to be used with an expanding force applied at different locations along the interior surface of the deformation region 232. The resulting deformation, can provide an offset or shifting in alignment of the anchoring regions affixed to the deformation region 232. Other forces may be used to create an expansion of the deformation region 232 from within to elongate the distance between the anchoring regions 202, 218 slightly, rather than reducing such distance and increasing the compressive force between the bone pieces.

Referring to FIG. 9, an exemplary deformation tool 300 can be provided in which a tip 302 is configured to be squeezed together to provide outward forces and/or torques to portions of the deformation regions described herein. For example, the tip portions of such a tool can be placed along interior corners or along other particular locations along the interior edges of the deformation region. Outwardly-directed forces can then be applied to the deformation region by squeezing the handles of the expanding-tip tool. A deformation tool having a configuration similar to that of a conventional pair of snap-ring pliers, for example, having two pivots between the handles and the tips, can also be used to produce an expanding force on portions of the deformation region of the exemplary bone plates described herein.

A plurality of notches, grooves, indentations, or the like can be created using the tool 300, as described above, to create indentations or protrusions. In other cases, notches, grooves, indentations, or the like can be pre-formed on the deformation region to facilitate alignment of the tool with certain locations along the deformation region.

In further configurations, an adjustable bone plate can be provided where the shape of the plate can be modified before and/or during the affixing process to better follow the shape or contours of the bone portions being joined or held in a particular orientation. Such bone plate includes two or more anchoring regions and one or more deformation regions, as described herein. A first anchoring region of the plate can be affixed to a first bone portion. The deformation region can then be deformed, for example, using one or more tools to provide compressive or expansive forces or torques to the deformation region. Such deformation can be configured to provide a better alignment of a second anchoring region with a second bone portion. Once satisfactory alignment has been achieved, the second anchoring region can be affixed to the second bone portion. Further steps of deformation and affixing can be performed if the bone plate is provided with two or more deformation regions. In this manner, the bone plate can be more precisely adjusted during the affixing process. Alternatively, such shaping of the bone plate by deforming the deformation region(s) may be performed prior to affixing the bone plate to a portion of bone. This device and procedure can provide better conformance of the bone plate to the bone portions than may be achieved with conventional bone plates, which generally require pre-shaping prior to affixing any portion of the plate to a bone portion.

The bone plates described herein may be configured for use on any suitable bone of the human body and/or of another vertebrate species. Exemplary bones may include bones of the arms (e.g., radius, ulna, humerus), legs (e.g., femur, tibia, fibula, patella), hands, feet, the vertebrae, scapulas, pelvic bones, cranial bones, the ribs and/or the clavicles, among others.

A typical bone plate such as described above can be formed with a thickness between about 1/16 of an inch and about 3/16 of an inch. A width of the anchoring regions, which may contain holes configured to receive fasteners therethrough, may be similar or may be between about ¼ inch and about ½ inch. A length of the deformation region along the longitudinal axis connecting the anchoring regions may be between about ½ inch and about 2 inches. The length of each anchoring region along this longitudinal axis may be between about ½ inch and about 3 inches. A width of the lateral sides of the deformation region can be between about 1/16 inch and about ⅛ inch. The overall width of the deformation region can preferably be somewhat wider than the width of the anchoring regions extending therefrom. Various edges of the bone plate can be provided with chamfers, for example, between about 0.01 inch and about 0.005 inches, with chamfer angles between about 30 and about 60 degrees.

These exemplary dimensions and geometric parameters can be varied for different applications. For example, such dimensions may be smaller if the bone plate is intended to be affixed to smaller bones such as finger bones, or larger for use on larger bones such as the femur or humerus. The materials and dimensions for a particular bone plate can be selected, for example, to provide both sufficient rigidity to support the bone portions and appropriate mechanical properties of the deformation region to facilitate plastic deformation and reshaping thereof while also providing sufficient rigidity to support the attached bone portions after such deformation has been performed. Portions of a bone plate can be formed or processed under different conditions to vary the resultant mechanical properties. For example, certain areas of the bone plate may be subjected to various heat treatments and/or mechanical work to increase or decrease local properties such as strength, toughness, various mechanical moduli, and the like. In certain configurations, the thicknesses and/or widths of various parts of the deformation and anchoring regions may vary within a single bone plate.

The present invention has been described in terms of one or more preferred embodiments, and it should be appreciated that many equivalents, alternatives, variations, and modifications, aside from those expressly stated, are possible and within the scope of the invention.

1. A method of setting a fracture in a bone, the method comprising:
securing a bone plate to portions of a bone on opposing sides of a fracture in the bone by affixing a plurality of fasteners through respective passages in the bone plate and into bone on the opposing sides of the fracture; and
with the bone plate secured to the bone, deforming at least one of a pair of bridge arms forming a central portion of the bone plate extending between portions of the bone plate affixed by the plurality of fasters on the opposing sides of the fracture to position the opposing sides of the fracture proximate one another to extend coaxially across the fracture.

2. The method of claim 1 wherein deforming the at least one of the pair of bridge arms includes at least one of bending and twisting at least a portion of the at least one of the pair of bridge arms.

3. The method of claim 1 wherein deforming the at least one of the pair of bridge arms includes changing a spaced relationship of the pair of bridge arms.

4. The method of claim 1 wherein deforming the at least one of the pair of bridge arms includes deforming the at least one of the pair of bridge arms to produce a compressive stress between the opposing sides of the fracture toward the fracture.

5. A bone plate system configured to be adjusted in vivo to secure proximate portions of a bone together, the bone plate system comprising:
a first anchoring region extending from a first end to a second end to form a substantially planar surface configured to extend coaxially with a first surface of a bone and having formed therein a plurality of passages;
a first plurality of fasteners configured to extend through the plurality of passages formed in the first anchoring region to secure the first anchoring region to the first surface of the bone;
a second anchoring region extending from a first end to a second end to form a substantially planar surface configured to extend coaxially with a second surface of the bone and having formed therein a plurality of passages;
a second plurality of fasteners configured to extend through the plurality of passages formed in the second anchoring region to secure the second anchoring region to the second surface of the bone;
a deformation region including a first bridging arm and a second bridging arm extending in a parallel, spaced relation from the second end of the first anchoring region to the second end of the second anchoring region to arrange the first anchoring region and the second anchoring region in a spaced relation and define a plane that is substantially co-planar with the first surface and the second surface; and
wherein the first bridging arm and the second bridging arm are configured to deform from the parallel, spaced relation forming the plane that is substantially co-planar with the first surface and the second surface after the first anchoring region is secured to the first surface of the bone by the first plurality of fasteners and the second anchoring surface is secured to the second surface of the bone by the second plurality of fasteners to perform simultaneous adjustments of the bone plate system and relative positions of the first surface of the bone and the second surface of the bone with respect to a fracture formed in the bone between the first surface of the bone and the second surface of the bone.

6. The system of claim 5 wherein the deformation region has reduced structural integrity compared to the first anchoring region and the second anchoring region.

7. The system of claim 5 wherein parallel, spaced relation between the first bridging arm and the second bridging arm has a shape of one of a rectangle, a square, a hexagon, a trapezoid, an octagon, an oval, and an ellipse, and a circle.

8. The system of claim 5 wherein first bridge arm and second bridge arm include at least one of a plurality of protrusions and a plurality of indentations configured to contact a deformation tool to facilitate deformation of the first bridge arm and the second bridge arm.

9. A bone plate configured to be deployed in vivo to secure proximate portions of a bone together, the bone plate comprising:
a first anchoring region, extending in at least two direction to form a surface configured to extend coaxially with a bone and having formed therein a plurality of passages configured to receive fasteners to secure the first anchoring region to a first portion of the bone;
a second anchoring region extending in at least two direction to form a surface configured to extend coaxially with the bone and having formed therein a plurality of passages configured to receive fasteners to secure the second anchoring region to a second portion of the bone;
a deformation region extending to join the first anchoring region and the second anchoring region along at least one plane extending substantially co-planar with the surface of the first anchoring region and the surface of the second anchoring region and having reduced structural integrity with respect to the first anchoring region and the second anchoring region to deform the deformation region and, thereby, adjust a relative orientation between the first anchoring region and the second region in response to forces applied to the deformation region.

10. The bone plate of claim 9 wherein the deformation region includes a first bridging arm and a second bridging arm extending in a parallel, spaced relation between the first anchoring region and the second anchoring region to arrange the first anchoring region and the second anchoring region in a spaced relation and wherein the first bridging arm and the second bridging arm are configured to deform from the parallel, spaced relation to perform simultaneous adjustments of relative positions of the first anchoring region and the second anchoring region.

11. The system of claim 10 wherein parallel, spaced relation between the first bridging arm and the second bridging arm forms a shape of one of a rectangle, a square, a hexagon, a trapezoid, an octagon, an oval, and an ellipse, and a circle.

12. The system of claim 10 wherein first bridge arm and second bridge arm include at least one of a plurality of protrusions and a plurality of indentations configured to contact a deformation tool to facilitate deformation of the first bridge arm and the second bridge arm.
